(19) United States
(12) Patent Application Publication
(10) Pub. No.: US 2004/0175426 A1
(43) Pub. Date: Sep. 9, 2004

(54) Controlled release of highly soluble agents

(75) Inventor: Paul Ashton, Boston, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/763,696

(22) Filed: Jan. 23, 2004

Related U.S. Application Data
(60) Provisional application No. 60/442,499, filed on Jan. 24, 2003.

(51) Int. Cl. A61K 9/24

(52) U.S. Cl. 424/471

(57) ABSTRACT

The invention provides for sustained release formulations for systemic delivery of drugs or other therapeutic agents that are highly soluble in water. Methods of administration of the agents and devices useful for administration are also disclosed. In particular, sustained release formulations are disclosed for the treatment of glaucoma, including methods and devices suitable for the administration of the formulations.

### BACKGROUND OF THE INVENTION

Numerous devices have been developed for local delivery of drugs within a living organism over a prolonged period of time. Such devices have the advantage of providing high concentrations of active compounds to the locus where their activity is desired, while maintaining low systemic concentrations of the same drug. In cases where the active compound is toxic, as is often true where the drugs are steroidal or anti-neoplastic compounds, maintaining low systemic concentrations is of great importance. Likewise, maintaining high local concentrations of drugs is important where the disease state can only be effectively treated with drugs at or near safe-dosaging limits.

Implantable drug compositions have been developed that deliver compounds in a single dose and provide controlled delivery of such compounds. In particular, in U.S. Pat. No. 6,051,576, Ashton et al. have described pharmaceutical compounds covalently linking two or more drug compounds (parent drugs) to form a single compound that has relatively low solubility in biological fluids, and that is quickly hydrolyzed to form the parent compounds when dissolved at or near pH 7.4. The Ashton et al. compounds are thus capable of providing high local concentrations of the parent drugs, while maintaining low systemic concentrations.

Despite the current capability of delivering high local concentrations, there is a need for an improved device that provides localized sustained-release drug delivery for agents that are readily soluble in physiological fluids. Many therapeutic agents are most readily available in the form of salts that are highly soluble in water and are, therefore, normally unsuited for formulation and delivery through commonly used sustained release systems.

### SUMMARY OF THE INVENTION

A sustained-release formulation is prepared with an inner core that contains a therapeutically effective amount of at least one agent and an outer polymer skin covering at least a portion of the inner core. In certain embodiments, the agent is a free base having a solubility in aqueous solution of about 10 mg/ml or less. In other embodiments, the agent is a charge-neutral protonated acid having a solubility in aqueous solution of about 10 mg/ml or less. Other embodiments of the invention are also described, including devices and methods for delivering the sustained-release formulation. In particular, a method for treating glaucoma is described.

### BRIEF DESCRIPTION OF THE FIGURES

FIG. 1 shows a release profile of albumin from a device coated with poly-(D,L-lactide-co-glycolide) and polyethylene glycol.

FIG. 2 shows a release profile of albumin from a device coated with polyethylene glycol.

### DETAILED DESCRIPTION OF THE INVENTION

The invention provides for sustained release formulations and devices for systemic delivery of drugs or other therapeutic agents that are highly soluble (as defined herein) in water. Such agents, when provided in free-base or protonated acid form, may be suitable for use in sustained-release therapeutic formulations, as well as methods and apparatuses for delivery of the formulations.

In preferred embodiments of the invention, the therapeutic agent is a free base that is provided, e.g., as a hydrophobic viscous oil. As used herein, the term “free base” means an agent with a basic nitrogen moiety that exists primarily in protonated (salt) form if the agent is dissolved in water. The free base has a conjugate acid with a pKa greater than about 4 and less than about 14, preferably greater than about 5 and less than about 12. Without limitation, moieties that typically include a basic nitrogen are amines, hydrazines, anilines, pyridines, amidines, and guandines.

In other embodiments, the therapeutic agent is a protonated acid. As used herein, the term “protonated acid” means an agent having a moiety capable of being deprotonated in aqueous solution to form a salt, where the moiety has a pKa greater than about 4 but less than about 14, preferably greater than about 5 but less than about 12. Without limitation, exemplary acidic moieties include carboxylate, phosphate, sulfonamide, thiol, imidazole, and imide.

The agent in its salt form (e.g. the unprotonated form of the protonated acid and the protonated form of the free-base) is preferably highly soluble in water, whereas the agent itself, e.g., protonated acid or free-base, preferably has a low solubility in water.

As discussed herein, an agent in free-base form is referred to as being in “uncharged” or “charge-neutral” form; when protonated, such an agent is referred to as being in “charged”, “protonated”, or “salt” form. Analogously, a protonated acid agent is referred to as being in “uncharged” or “charge-neutral” form; in its deprotonated form, such an agent is referred to as being in “charged”, “deprotonated”, or “salt” form.

At least a portion of the agent is incorporated into a biocompatible (i.e., biologically tolerated) polymer vehicle, also referred to synonymously herein as a polymer skin, a tube, or a coating. In some embodiments, the agent is present as a plurality of granules dispersed within the polymer vehicle. In such cases, it is preferred that the agent be relatively insoluble in the polymer vehicle; however, the agent may possess a finite solubility coefficient with respect to the polymer vehicle and still be within the scope of the present invention. In any event, the solubility of the agent in the polymer vehicle should be such that a portion of the agent will disperse throughout the polymer vehicle, while the remainder of the agent continues to exist in substantially granular form.

In some embodiments, it is preferred that the polymer vehicle be a relatively non-polar or hydrophobic polymer which acts as a good solvent for the relatively hydrophobic agent. In such cases, the solubility of the agent in the polymer vehicle should be such that the agent will dissolve thoroughly in the polymer vehicle, being distributed homogeneously throughout the polymer vehicle.

Without wishing to be bound by any particular mechanism, it is expected that release of a free-base agent occurs at a given physiologic site as the free base diffuses from the inner core and becomes protonated in the physiological fluid. Upon protonation, the agent dissolves in the surrounding fluid. In embodiments utilizing a protonated acid, it is expected that the release of the agent occurs as the acid diffuses from the inner core and becomes deprotonated in the physiological fluid, whereupon the agent dissolves rapidly into the fluid. In either embodiment, it is expected that the rate of release of the agent is controlled more by the rate of ionization of the agent (e.g., rate of protonation of the free base or rate of deprotonation of the protonated acid) than by the rate of the agent's diffusion from the inner core or the rate of the charged agent's dissolution in the immediately surrounding fluid.

In certain embodiments, the vehicle or coating may be formed with the agent as a substantially homogeneous system, formed by mixing one or more suitable monomers and a suitable agent, then polymerizing the monomer to form the polymer system. In this way, the agent is dissolved or dispersed in the polymer. In other embodiments, the agent is mixed into a liquid polymer or polymer dispersion and then the polymer is further processed to form the inventive coating. Suitable further processing may include crosslinking with suitable crosslinking agents, further polymerization of the liquid polymer or polymer dispersion, copolymerization with a suitable monomer, block copolymerization with suitable polymer blocks, etc. The further processing traps the agent in the polymer so that the agent is suspended or dispersed in the polymer vehicle.

In certain embodiments, the agent is admixed with a polymer matrix prior to the administration of the skin. The polymer matrix material is selected so as to not destabilize the agent. Preferably, the matrix material is selected so that sustained release of the agent is controlled by the rate of protonation of the free-base agent, or by the rate of deprotonation of the protonated acid where applicable, such that the agent's diffusion through the matrix has little or no effect on the agent's release rate from the matrix.

According to the invention, the materials selected for use in the polymer matrix and/or in the polymer vehicle are selected to be stable during the release period for the drug delivery device. In other embodiments, bioerodible materials may be used so that the device erodes in situ after it has released the agent for a predetermined amount of time. In either case, the materials are preferably selected so that, for the desired life of the delivery device, the materials are stable and do not significantly erode, and the pore size and permeability of the device do not change.

The polymer material used for the matrix, the vehicle, or both, may be formed from a single polymer or a mixture of polymers. In certain embodiments, the material is a mixture that includes at least two polymers or copolymers having different molecular weights. In certain embodiments, the polymer mixture includes a copolymer having different monomeric units, wherein the molar ratio of different monomeric units is not 1:1.

In certain embodiments, the polymer used as the matrix or as the vehicle has a melting temperature above 40° C., preferably above about 45° C., more preferably above 50° C., and most preferably above 55° C.

According to the invention, the inner core may include one or more pharmaceutically active agents; preferably, where two or more agents are used, the agents should not have competing states of protonation. For example, where two agents are used and one is a free base, the other should not be a protonated acid; similarly, where one agent is a protonated acid, the other agent(s) should not be a free base. However, a free base may optionally be combined with other free base(s), and a protonated acid may optionally be combined with other protonated acid(s). Any given free base or protonated acid may also be combined with one or more agents that are not readily protonated or deprotonated.

In cases where the agent(s) are admixed with a polymer matrix, one or more polymer materials may be used. Other materials may also be used in connection with the inner core, including lipids (including long chain fatty acids) and waxes, anti-oxidants, and release modifiers (e.g., water). These materials may be biocompatible and may remain stable during the manufacturing process, e.g., during the extrusion process.

The polymers or other biomaterials admixed as part of the matrix in the inner core may be selected so that the release rate of an agent from the matrix is determined, at least in part, by the physico-chemical properties of the agent, and not by the properties of the matrix. In certain embodiments, the pH of the matrix may be selected such that it modifies the release rate of the agent. For example, where the agent is a free base, the matrix may include basic moieties, e.g., having a pKa that is higher than that of the agent, thereby slowing the protonation rate and ultimately the release rate of the agent. The matrix may also have moieties having a pKa that is less than but relatively close to that of the free base agent. In either of such embodiments the matrix functions as a buffer to the protonation of the free base agent and, ultimately, to its release from the device. In addition, the pH microenvironment of the matrix may be varied by the addition of basic additives or by the use of phosphate or other standard buffers, thereby controlling the protonation of the agent and its diffusion from the matrix.

Analogously, in embodiments utilizing a protonated acid agent, the matrix may include acidic moieties, e.g., having a pKa that is lower than that of the protonated acid. The matrix may also have a pKa that is greater than but relatively close to that of the protonated acid agent. In either of such embodiments, the matrix functions as a buffer to the ionization of the protonated acid agent and, ultimately, to its release from the device. In addition, acidic additives or phosphate or other standard buffers may also be used to vary the pH microenvironment of the matrix, thereby controlling the protonation of the agent and its release from the matrix.

In certain embodiments, the polymer material for the matrix, vehicle, or both, is chosen so as to assist in controlling the release rate of the agent. In certain embodiments, the polymer may be a polymer mixture having more than one polymer or a copolymer of two or more monomers, wherein the release rate of the agent is determined, at least in part, by the molar ratio of polymers or of the copolymer monomers in the vehicle or the matrix. In certain embodiments, the release rate of the agent can be further modified with non-polymeric additives to the polymer that alter the chemical properties of the matrix or the vehicle.

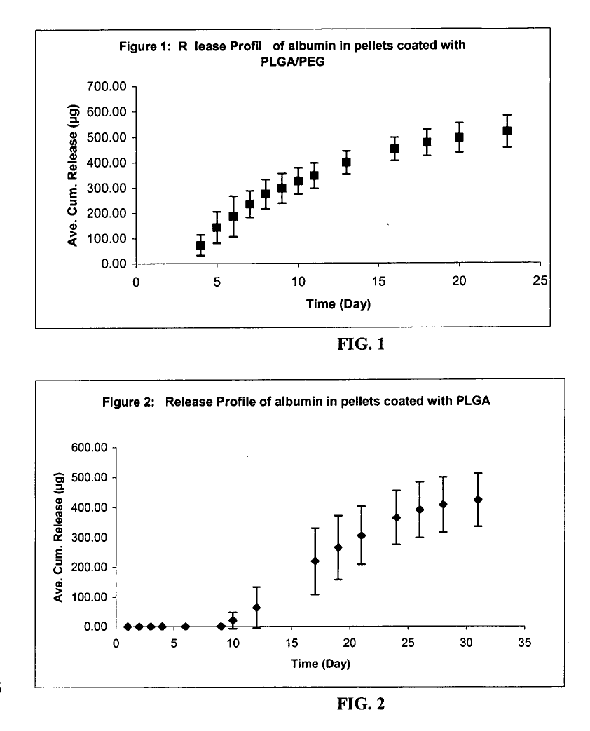

FIGS. 1 and 2 illustrate examples of release profiles of agents from a polymer mixture (FIG. 1) and a single polymer (FIG. 2).

In certain embodiments, the agent(s) are prepared for sustained release into intradermal, intramuscular, intraperitoneal, or subcutaneous sites. For instance, the agent may be formulated in a polymer or hydrogel which may be introduced at a site in the body where it remains reasonably dimensionally stable and localized for at least a period of days, and more preferably for 2-10 weeks or more. In other embodiments, the highly water-soluble agent(s) may be provided in a sustained-release device, which in turn can be implanted at one or more particular positions in the body, preferably where (by the nature of the position or by means of securing the device) it is not likely to migrate substantially from the position or compartment into which it is implanted.

Another aspect of the invention provides a pharmaceutical package including one or more highly water-soluble agent(s) formulated for sustained release (such as in a sustained-release device), and associated with instructions or a label for use in patients.

Exemplary agents that are highly soluble in their salt forms but have low solubility in their respective free-base or protonated acid forms include timolol maleate, betaxolol hydrochloride, metformin hydrochloride, vancomycin hydrochloride, erythromycin lactobionate, ranitidine hydrochloride, sertraline hydrochloride, ticlopidine hydrochloride, nicotine bitartrate, oxybutynin hydrochloride, cefuroxine axetil, and salts of captopril, tramadol, diltiazem, propanolol, amoxicillin, cefaclor, clindamycin, azithromycin, ceftazidine, and certain carbonic anhydrase inhibitors (e.g., dorzolamide hydrochloride, acetazolamide, brinzolamide, methazolamide, and dichlorphenamide). Other agents such as proteins, peptides or derivatives thereof may also be suitable.

In certain embodiments, the solubility in water of the uncharged form of the agent is less than 10 mg/ml, or even less than 1.0 mg/ml, 0.1 mg/ml, 0.01 mg/ml or 0.001 mg/ml.

In certain embodiments, the agent in its salt form is at least 10 times more soluble in water relative to the uncharged form, or even at least 100, 1000 or preferably 10,000 times more soluble in water relative to the uncharged form of the agent.

In certain other embodiments, when disposed in biological fluid (such as serum, synovial fluid, cerebrospinal fluid, lymph, urine, etc), the sustained-release formulation provides sustained release of the therapeutically active form of an agent for a period of at least 24 hours, and over that period of release the concentration of the agent in fluid outside the polymer skin is less than 10% of the concentration of the agent inside the polymer skin, and even more preferably less than 5%, 1% or even 0.1% of the concentration of the agent inside the polymer skin.

In other embodiments, the subject invention provides methods and compositions for treating or reducing the risk of disease or other physiological conditions, such as glaucoma. The invention particularly contemplates sustained-release compositions for systemic delivery of therapeutic agents that are highly water-soluble in their salt forms. In preferred embodiments, such highly water-soluble agents include anti-glaucoma agents such as betaxolol hydrochloride or timolol maleate, or certain carbonic anhydrase inhibitors, e.g. acetazolamide.

By “sustained-release device” or “sustained-release formulation” it is meant a device or formulation that releases an agent over an extended period of time in a controlled fashion. Examples of sustained-release devices and formulations suitable for the present invention may be found in, for example, U.S. Pat. No. 6,375,972, U.S. Pat. No. 5,378,475, U.S. Pat. No. 5,773,019, and U.S. Pat. No. 5,902,598. For example, sustained-delivery devices might include, but are not limited to sutures, stents, surgical screws, prosthetic joints, artificial valves, plates, pacemakers, etc.

In another aspect of the invention, a device coated with an agent and a polymer matrix may be applied to surgical implements such as screws, plates, washers, sutures, prosthesis anchors, tacks, staples, electrical leads, valves, membranes. The device may be a catheter, implantable vascular access ports blood storage bag, blood tubing, central venous catheter, arterial catheter, vascular graft, intraaortic balloon pump, heart valve, cardiovascularsuture, artificial heart, a pacemaker, ventricular assist pump, extracorporeal device, blood filter, hemodialysis unit, hemoperfasion unit, plasmapheresis unit, and filter adapted for deployment in a blood vessel.

In addition, the co-extrusion methods of forming a sustained-release drug delivery device disclosed in by Chou, et al., under U.S. Provisional Application No. 60/377,974, filed May 7, 2002, and U.S. Provisional Application No. 60/437,576, filed Dec. 31, 2002, are applicable to the present invention and may optionally be used. Each of the above specifications is incorporated in its entirety here.

According to the present invention, a sustained-release device includes an inner core or reservoir that includes a therapeutically effective agent, and a tube (also synonymously referred to herein as a skin, vehicle, or a first coating layer) which encloses at least a portion of the core. The agent is present in its uncharged form, rather than in its salt form. The inner core may optionally include a polymer matrix interspersed with the agent. In certain embodiments, the tube and/or the polymer matrix may optionally be permeable, semi-permeable, or impermeable to the agent. Preferably, the tube is an impermeable polymer skin and covers at least a portion of the core but leaves at least a portion of the core uncovered, thereby facilitating the release of the agent from the uncovered portion and not from the covered portion.

Thus, the tube is impermeable to the agent in preferred embodiments. In other embodiments the tube may be semi-permeable or permeable to the agent. The tube may also optionally contain a member, such as a disc, at one or both ends of the tube wherein the member is optionally permeable, semi-permeable, or impermeable to the agent. In particular embodiments, an impermeable disc is used at one end of the tube, and a permeable or semi-permeable member is used at the other end of the tube, thereby facilitating release of the agent from one end of the tube. In preferred embodiments, the tube is a polymeric coating layer.

In still other embodiments the device may include one or more additional coating layers that are optionally permeable, semi-permeable, or impermeable to the agent. For instance, the tube may form a first coating layer that is impermeable to the passage of the agent and covers a portion of the inner core, leaving a portion of the core uncoated. A second layer that is permeable or semi-permeable to the agent may optionally be used to coat both the first layer and the inner core. In such embodiments, the agent passes from the uncoated inner core portion and through the second coating layer but not through the first layer.

The portion of the inner core coated by the tube may be increased or decreased, thereby increasing or decreasing the rate of passage of the agent from the core. In embodiments utilizing multiple layers, an impermeable third coating layer may be used to cover at least a portion of a permeable or semi-permeable second layer, thereby enabling more precise control of the release rate of the agent. In such embodiments, the third coating layer may be selected so as to slow the release of the agent from the inner core into contact with a mammalian organism, e.g., a human. The third coating layer need not provide gradual release or control of the agent into the biological environment; however, the third coating layer may be advantageously selected to have that property or feature.

Depending on the desired delivery rate of the agent, the tube may coat only a small portion of the surface area of the inner core for faster release rates of the agent or may coat large portions of the surface area of the inner core for slower release rates of the agent.

For faster release rates, the tube may coat up to 10% of the surface area of the inner core. In certain embodiments, approximately 5-10% of the surface area of the inner core is coated with the tube for faster release rates. In some embodiments, less than 5% may be coated; in other embodiments less than 1% may be coated, or even less than 0.1% may be coated.

For slower release rates, the tube may coat at least 10% of the surface area of the inner core. Preferably, at least 25% of the surface area of the inner core is coated with the first coating layer. For even slower release rates, at least 50% of the surface area may be coated. For even slower release rates, at least 75% of the surface area may be coated. For even slower release rates, at least 95% of the surface area may be coated.

Thus, any portion of the surface area of the inner core up to but not including 100% may be coated with a first impermeable coating layer to achieve the desired rate of release of the agent. In preferred embodiments, coating less than 100% of the core is desired so as to allow the agent to release; preferably, coating of less than about 97% is desired. In some embodiments, about 10% or more of the core may remain uncoated by the skin. In other embodiments, the uncoated portion is about 25% or more, or even about 50%, 75%, or 90% or more.

The first coating may be positioned anywhere on the inner core, including but not limited to the top, bottom or any side of the inner core. In addition, it could be on the top and a side, or the bottom and a side, or the top and the bottom, or on opposite sides or on any combination of the top, bottom or sides.

The composition of the first layer, e.g., a polymer, is selected so as to allow the above-described controlled release. The preferred composition of the first layer may vary depending on such factors as the active agent, the desired rate of release of the agent and the mode of administration. The identity of the active agent is important because its molecular size determines, at least in part, its rate of release into the second layer.

In still other embodiments, the first coating layer is semi-permeable or permeable to the agent while a second layer is impermeable and covers a portion of the device.

The materials chosen for the inner core matrix, the tube, and/or any additional layers are selected to be non-bioerodible, such that they are stable during the formation of the device and during the release period for the device. In other embodiments, the polymer matrix or the tube or additional layers may optionally be bioerodible.

Once implanted, the device gives a continuous supply of the agent to internal regions of the body without requiring additional invasive penetrations into these regions. Instead, the device remains in the body and serves as a continuous source of the agent to the affected area. In another embodiment, the device further comprises a means for attachment, such as an extension of a non-erodible polymer coating layer, a backing member, a support ring, suture tab, or suture.

A non-bioerodible polymer coating layer may completely or partially cover the inner core. In this regard, any portion of the surface area of the inner core up to and including 100% may be coated with the polymer coating layer as long as the pellet is protected against disintegration, prevented from being physically displaced from its required site, and as long as the polymer coating layer does not unduly retard the release rate. In other embodiments the polymer coating layer may be bioerodible, provided that its decomposition in a physiological environment does not adversely affect the release rate of the agent.

A number of polymers may be used to construct the devices of the present invention. Such polymers are preferably inert, non-immunogenic, and/or of the desired permeability.

Materials that may be suitable for fabricating the device include naturally occurring or synthetic materials that are biologically compatible and essentially insoluble in body fluids with which the material will come in contact. Rapidly dissolving materials or materials highly soluble in fluids are not desirable, since dissolution of the wall would affect the constancy of the drug release, as well as the capability of the system to remain in place for a prolonged period of time.

Naturally occurring or synthetic materials that are biologically compatible and essentially insoluble in body fluids which the material will come in contact include, but are not limited to, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerate copolymer, silicone rubbers, especially medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitrile copolymer.

Specifically, the device of the present invention may be made of any of the above-listed polymers or any other polymer which is biologically compatible, essentially insoluble in body fluids with which the material will come in contact, and essentially impermeable to the passage of the effective agent. The term “impermeable,” as used herein, means that the layer will not allow passage of the effective agent at a rate required to obtain the desired local or systemic physiological or pharmacological effect.

In embodiments utilizing one or more impermeable layers, including an impermeable disc, the impermeable portion may be positioned anywhere over the inner core and/or other coating layers, including but not limited to the top, bottom or any side of a first permeable or semi-permeable coating layer and an inner core. In addition, the impermeable layer could be placed on the top and a side of the device, or the bottom and a side, or the top and the bottom, or on opposite sides or on any combination of the top, bottom or sides.

In certain embodiments, one or more layers are used, and each layer is preferably biologically compatible and essentially insoluble in body fluids which the device will come in contact.

According to the present invention, the agent diffuses in the direction of lower chemical potential, i.e., toward the exterior surface of the device. At the exterior surface of the device, equilibrium is again established. In certain embodiments using multiple coating layers, when the conditions on both sides of the outer coating layer are maintained constant, a steady state flux of the effective agent will be established in accordance with Fick's Law of Diffusion. The rate of passage of the drug through the material by diffusion is generally dependent on the solubility of the drug therein, as well as on the thickness of the wall. This means that selection of appropriate materials for fabricating the tube or first layer will be dependent on the particular drug to be used.

The rate of diffusion of the effective agent through a polymeric layer of the present invention may be determined via diffusion cell studies carried out under sink conditions. In diffusion cell studies carried out under sink conditions, the concentration of drug in the receptor compartment is essentially zero when compared to the high concentration in the donor compartment. Under these conditions, the rate of drug release is given by:

Q/t=(D·K·A·dC)/h

where Q is the amount of drug released, t is time, D is the diffusion coefficient, K is the partition coefficient, A is the surface area, dC is the difference in concentration of the drug across the membrane, and h is the thickness of the membrane.

In the case where the agent diffuses through a layer via water-filled pores, there is no partitioning phenomenon. Thus, K is eliminated from the equation. Under sink conditions, if release from the donor side is very slow, the value dC is essentially constant and equal to the concentration of the donor compartment. The release rate therefore becomes dependent on the surface area (A), thickness (h) and diffusivity (D) of the membrane. In the construction of the device of the present invention, the size (and therefore surface area) of the membrane is mainly dependent on the molecular size of the effective agent.

Thus, permeability values may be obtained from the slopes of a Q versus time plot. The permeability P is related to the diffusion coefficient D by:

P=(K·D)/h

Once the permeability is established for the coating permeable to the passage of the agent, the surface area may be determined of the area that is coated with the coating impermeable to the passage of the agent. This is done by progressively reducing the available surface area until the desired release rate is obtained.

Exemplary microporous materials suitable for use as permeable or semi-permeable coating layer material, for instance, are described in U.S. Pat. No. 4,014,335 which is incorporated herein by reference in its entirety. These materials include cross-linked polyvinyl alcohol, polyolefins or polyvinyl chlorides or cross-linked gelatins; regenerated, insoluble, nonerodible cellulose, acylated cellulose, esterified celluloses, cellulose acetate propionate, cellulose acetate butyrate, cellulose acetate phthalate, cellulose acetate diethyl-aminoacetate; polyurethanes, polycarbonates, and microporous polymers formed by co-precipitation of a polycation and a polyanion modified insoluble collagen. Cross-linked polyvinyl alcohol is preferred.

The devices of the invention may be made in a wide variety of ways, such as by obtaining an effective amount of the agent and compressing the agent to a desired shape. Once shaped, a first coating layer may be applied by dipping the device one or more times in a solution containing the desired polymer. Optionally, the first coating may be applied by dropping, spraying, brushing or otherwise coating the outer surface of the device with the polymer solution. When using a polyvinyl alcohol solution to obtain the second coating layer, the desired thickness may be obtained by applying several coats. Each coat may be dried prior to applying the next coat. Finally, the device may be heated to adjust the permeability of the outer coating.

In certain embodiments, one or more impermeable discs may be applied directly over a semi-permeable or permeable layer before coating with an impermeable polymer layer. In the case of a cylindrical core, an impermeable film may be wrapped around the core after discs are applied to one or both ends. Thus, a second coating layer is optionally used wherein the second layer includes both an impermeable film and impermeable discs.

An impermeable polymer layer should be thick enough to substantially prevent release of the agent across it other than across the area not covered (the diffusion layer or port). Due to the desirability of minimizing the size of an implant, the thickness of an impermeable film layer therefore may be 0.01 to 2 millimeters, preferably 0.01 to less than 0.5 millimeters.

Similarly, the impermeable disc should also be thick enough to substantially prevent drug release except through a specifically prepared membrane or port. Due to the desirability of minimizing the size of an implant, the thickness of the impermeable disc may be from 0.01 to 2 millimeters, preferably from 0.01 to less than 1 millimeter.

In certain other embodiments, a sustained-release device is formed by co-extruding an inner core containing a therapeutically active agent with a self-supportable outer layer or tube. The core is optionally also formed by admixing the therapeutically active agent with a polymer matrix prior to formation of the device, thereby increasing the viscosity of the core and enhancing its extrudability. The agent (and matrix where utilized) thereby forms the inner core of the sustained-release delivery device, which may be formed according to the invention more fully described by Chou, et al, in the U.S. Provisional Application No. 60/377,974, and in U.S. Provisional Application No. 60/437,576, the teachings of which are hereby incorporated in their entirety by reference. The device is preferably tube-shaped, although products with other cross sections may be prepared. More than one active agent is also optionally includable in the inner core, enabling the sustained release of multiple agents.

The extrusion process, when applied, has the ability to deliver the co-extruded core/matrix materials at pressures and flow rates sufficient to form the product of sufficient size that may be implanted into a patient. In some embodiments, the device is of a size that may be implanted into the eye, including without limitation implanting it subconjunctivally into the sclera of the eye.

As discussed herein, low solubility agents are those having solubility in aqueous solution of about 10 mg/ml or less.

The solubility of the therapeutic agents may also be discussed in terms of LogP, where high solubility agents typically have LogP values of about 4 or less, moderate solubility agents typically have LogP values greater than about 4 and less than about 7, and low solubility agents have LogP values of about 7 or greater. As used herein, an agent's “LogP” refers to the logarithm of P (Partition Coefficient), where P is a measure of how well a substance partitions between octanol and water. P itself is a constant, defined as the ratio of concentration of compound in aqueous phase to the concentration of compound in octanol according to the following:

Partition Coefficient, P=[Organic]/[Aqueous] where [ ]=concentration

LogP=log₁₀(Partition Coefficient)=log₁₀P

In practice, the LogP value will vary according to the conditions under which it is measured and the choice of partitioning solvent. A LogP value of 1 means that the concentration of the compound is ten times greater in the organic phase than in the aqueous phase. The increase in a LogP value of 1 indicates a ten-fold increase in the concentration of the compound in the organic phase as compared to the aqueous phase. Thus, a compound with a LogP value of 3 is 10 times more soluble in water than a compound with a LogP value of 4 and a compound with a LogP value of 3 is 100 times more soluble in water than a compound with a LogP value of 5.

In certain embodiments of the present invention, the agent in its salt form may have a LogP value of about 5 or less; in other embodiments the LogP value of the salt may be 4 or less, or even 3 or less in other embodiments. In certain embodiments, the agent in its uncharged form may have a LogP of about 5 or greater; in other embodiments the LogP of the uncharged agent may be about 7 or greater, or even about 9 or greater.

The phrase “pharmaceutically acceptable carrier” as used herein means a pharmaceutically acceptable material, composition or vehicle, such as a liquid or solid filter, diluent, excipient, solvent or encapsulating material, involved in carrying or transporting a subject drug from one organ, or portion of the body, to another organ, or portion of the body. Each carrier must be “acceptable” in the sense of being compatible with other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The above description of the sustained-release system and accompanying devices is merely illustrative of the invention and should not be considered as limiting the scope of the invention in any way, as various suitable compositions are well known by those skilled in the art. In particular, the methods of making the invention depend on the identity of the active agent and polymers selected. Given the active agent, the composition of a first coating, a second coating (the film and disc), or even a third coating if desired, one skilled in the art could easily make the devices of the present invention using conventional coating techniques.

### EXAMPLES

The invention can be more fully understood with reference to the following non-limiting examples.

### Example 1

A pellet (1.5 mm in diameter, 2.0 mg) containing poly-(D,L-lactide-co-glycolide)(PLGA) and bovine albumin (1:1,w:w) was hand compressed. The pellet was then dip coated in PLGA/PEG (polyethylene glycol) solution in acetone and air-dried. The dried coated pellet was then inserted in and covered by a silicone array, leaving a hole of 0.59 mm diameter uncovered by the silicone to permit the diffusion of the albumin.

The pellet was placed in 1.0 ml of phosphate buffer (pH 7.4), and its release tested at 37° C. Samples were taken periodically and analyzed for albumin by HPLC.

FIG. 1 shows the cumulative release profile for pellets coated with PLGA/PEG (8/2).

### Example 2

A pellet (1.5 mm in diameter, 2.0 mg) containing poly-(D,L-lactide-co-glycolide) (PLGA) and bovine albumin (1:1,w:w) was hand compressed. The pellet was then dip-coated in PLGA solution in acetone and air-dried. The dried coated pellet was then inserted in and covered by a silicone array, leaving a hole of 0.59 mm diameter uncovered by the silicone to permit the diffusion of the albumin.

The pellet was placed in 1.0 ml of phosphate buffer (pH 7.4), and its release tested at 37° C. Samples were taken periodically and analyzed for albumin by HPLC.

FIG. 2 shows the cumulative release profile for pellets coated with PLGA.

What is claimed is:

1. A sustained-release formulation, comprising:
an inner core comprising a therapeutically effective amount of at least one agent, wherein the at least one agent is optionally a free base or a protonated acid, said at least one agent having a solubility in aqueous solution of about 10 mg/ml or less; and
an outer polymer skin covering at least a portion of the inner core.

2. The formulation of claim 1, wherein the inner core further comprises a polymer matrix admixed with the at least one agent.

3. The formulation of claim 1 or 2, wherein the outer polymer skin is polycaprolactone (PCL), poly(vinyl acetate) (PVAC), poly(ethylene-co-vinyl acetate) (EVA), or poly(di-lactide-co-glycolide) (PLGA).

4. The formulation of claim 2, wherein the polymer matrix is polycaprolactone (PCL), poly(vinyl acetate) (PVAC), poly(ethylene-co-vinyl acetate) (EVA), or poly(di-lactide-co-glycolide) (PLGA).

5. The formulation of claim 1 or 2, wherein the outer polymer skin is non-bioerodible.

6. The formulation of claim 5, wherein the non-bioerodible polymer skin is selected from polyurethane, polysilicone, poly(ethylene-co-vinyl acetate), polyvinyl alcohol, and derivatives and copolymers thereof.

7. The formulation of claim 2, wherein the polymer matrix is non-bioerodible.

8. The formulation of claim 7, wherein the non-bioerodible polymer matrix is selected from polyurethane, polysilicone, poly(ethylene-co-vinyl acetate), polyvinyl alcohol, and derivatives and copolymers thereof.

9. The formulation of claim 2, wherein the polymer matrix is bioerodible.

10. The formulation of claim 9, wherein the bioerodible polymer matrix is selected from polyanhydride, polylactic acid, polyglycolic acid, polyorthoester, polyalkylcyanoacrylate, and derivatives and copolymers thereof.

11. The formulation of claim 1 or 2, wherein the outer polymer skin is bioerodible.

12. The formulation of claim 1 or 2, wherein the outer polymer skin is bioerodible and is selected from polyanhydride, polylactic acid, polyglycolic acid, polyorthoester, polyalkylcyanoacrylate, and derivatives and copolymers thereof.

13. The formulation of claim 1 or 2, wherein sustained release of the at least one agent occurs for a period of at least 24 hours and, over that period of release, the concentration of the at least one agent in fluid immediately surrounding the polymer skin is less than 10% of the concentration of the at least one agent in the inner core.

14. The formulation of claim 1 or 2, wherein sustained release of the at least one agent occurs for a period of at least 24 hours and, over that period of release, the concentration of the at least one agent in fluid immediately surrounding the polymer skin is less than 5% of the concentration of the at least one agent in the inner core.

15. The formulation of claim 1 or 2, wherein sustained release of the at least one agent occurs for a period of at least 24 hours and, over that period of release, the concentration of the at least one agent in fluid immediately surrounding the polymer skin is less than 1% of the concentration of the at least one agent in the inner core.

16. The formulation of claim 1 or 2, wherein sustained release of the at least one agent occurs for a period of at least 24 hours and, over that period of release, the concentration of the at least one agent in fluid immediately surrounding the polymer skin is less than 0.1% of the concentration of the at least one agent in the inner core.

17. The formulation of claim 1 or 2, wherein the at least one agent and the outer polymer skin are co-extruded.

18. The formulation of claim 2, wherein the polymer matrix comprises a basic moiety having a pKa higher than the pKa of the at least one agent, or an acidic moiety having a pKa lower than the pKa of the at least one agent.

19. The formulation of claim 1 or 2, wherein the outer polymer skin comprises a basic moiety having a pKa higher than the pKa of the at least one agent, or an acidic moiety having a pKa lower than the pKa of the at least one agent.

20. The formulation of claim 1, wherein the outer polymer skin is impermeable to the at least one agent and covers less than 100% of the inner core.

21. The formulation of claim 1, wherein the outer polymer skin is impermeable to the at least one agent and covers about 75% or less of the inner core.

22. The formulation of claim 1, wherein the outer polymer skin is impermeable to the at least one agent and covers about 50% or less of the inner core.

23. The formulation of claim 1 or 2, wherein the at least one agent in its salt form is selected from timolol maleate, betaxolol hydrochloride, metformin hydrochloride, vancomycin hydrochloride, captopril, erythromycin lactobionate, ranitidine hydrochloride, sertraline hydrochloride, tramadol hydrochloride, ticlopidine hydrochloride, nicotine bitartrate, oxybutynin hydrochloride, diltiazem hydrochloride, propanolol, amoxicillin, cefuroxine axetil, cefaclor, clindamycin, azithromycin, ceftazidine, dorzolamide hydrochloride, acetazolamide, brinzolamide, methazolamide, and dichlorphenamide.

24. A drug delivery device comprising:
a substrate having a surface, and
a sustained-release formulation adhered to the surface, the sustained release formulation comprising an inner core having a therapeutically effective amount of at least one agent, wherein the at least one agent is a free base or a protonated acid, and has solubility in water that is about 10 mg/ml or less, and an outer polymer skin covering at least a portion of the inner core.

25. The device of claim 24, wherein the inner core further comprises a polymer matrix admixed with the at least one agent.

26. The device of claim 24 or 25, wherein the outer polymer skin is impermeable to the at least one agent and covers less than 100% of the inner core.

27. The device of claim 24 or 25, wherein the outer polymer skin is polycaprolactone (PCL), poly(vinyl acetate) (PVAC), poly(ethylene-co-vinyl acetate) (EVA), or poly(di-lactide-co-glycolide) (PLGA).

28. The device of claim 25, wherein the polymer matrix is polycaprolactone (PCL), poly(vinyl acetate) (PVAC), poly(ethylene-co-vinyl acetate) (EVA), or poly(di-lactide-co-glycolide) (PLGA).

29. The device of claim 24 or 25, wherein the outer polymer skin is non-bioerodible.

30. The device of claim 24 or 25, wherein the outer polymer skin is non-bioerodible and is selected from polyurethane, polysilicone, poly(ethylene-co-vinyl acetate), polyvinyl alcohol, and derivatives and copolymers thereof.

31. The device of claim 25, wherein the polymer matrix is non-bioerodible.

32. The device of claim 31, wherein the non-bioerodible polymer matrix is selected from polyurethane, polysilicone, poly(ethylene-co-vinyl acetate), polyvinyl alcohol, and derivatives and copolymers thereof.

33. The device of claim 25, wherein the polymer matrix is bioerodible.

34. The device of claim 33, wherein the bioerodible polymer matrix is selected from polyanhydride, polylactic acid, polyglycolic acid, polyorthoester, polyalkylcyanoacrylate, and derivatives and copolymers thereof.

35. The device of claim 24 or 25, wherein the outer polymer skin is bioerodible.

36. The device of claim 24 or 25, wherein the outer polymer skin is bioerodible and is selected from polyanhydride, polylactic acid, polyglycolic acid, polyorthoester, polyalkylcyanoacrylate, and derivatives and copolymers thereof.

37. The device of claims 24 or 25, wherein sustained release of the at least one agent occurs for a period of at least 24 hours and, over that period of release, the concentration of the at least one agent in fluid outside the polymer skin is less than 10% of the concentration of the at least one agent in the polymer skin.

38. The device of claim 24 or 25, wherein sustained release of the at least one agent occurs for a period of at least 24 hours and, over that period of release, the concentration of the at least one agent in fluid outside the polymer skin is less than 5% of the concentration of the at least one agent in the polymer skin.

39. The device of claim 24 or 25, wherein sustained release of the at least one agent occurs for a period of at least 24 hours and, over that period of release, the concentration of the at least one agent in fluid outside the polymer skin is less than 1% of the concentration of the at least one agent in the polymer skin.

40. The device of claim 24 or 25, wherein the at least one agent and the outer polymer skin are co-extruded.

41. The device of claim 25, wherein the polymer matrix comprises a basic moiety having a pKa higher than the pKa of the at least one agent, or an acidic moiety having a pKa lower than the pKa of the at least one agent.

42. The device of claim 24 or 25, wherein the outer polymer skin comprises a basic moiety having a pKa higher than the pKa of the at least one agent, or an acidic moiety having a pKa lower than the pKa of the at least one agent.

43. The device of claim 24 or 25, wherein the at least one agent in its salt form is selected from timolol maleate, betaxolol hydrochloride, metformin hydrochloride, vancomycin hydrochloride, captopril, erythromycin lactobionate, ranitidine hydrochloride, sertraline hydrochloride, tramadol, ticlopidine hydrochloride, nicotine bitartrate, oxybutynin hydrochloride, diltiazem, propanolol, amoxicillin, cefuroxine axetil, cefaclor, clindamycin, azithromycin, ceftazidine, dorzolamide hydrochloride, acetazolamide, brinzolamide, methazolamide, and dichlorphenamide.

44. A method of providing sustained-release administration of a highly soluble agent, comprising:
providing a therapeutically effective amount of at least one agent, wherein said at least one agent is either a free base or a protonated acid, said at least one agent having a solubility in water that is about 10 mg/ml or less,
forming a sustained-release formulation having an inner core comprising the at least one agent, wherein the inner core is covered at least in part by an outer polymer skin;
providing the sustained-release formulation in a pharmaceutically acceptable carrier; and
administering the sustained-release formulation to a patient.

45. The method of claim 44, wherein the inner core further comprises the at least one agent dispersed in a polymer matrix.

46. The method of claim 44 or 45, wherein the outer polymer skin is impermeable to the at least one agent and covers less than 100% of the inner core.

47. The method of claim 44 or 45, wherein the outer polymer skin is polycaprolactone (PCL), poly(vinyl acetate) (PVAC), poly(ethylene-co-vinyl acetate) (EVA), or poly(di-lactide-co-glycolide) (PLGA).

48. The method of claim 45, wherein the polymer matrix is polycaprolactone (PCL), poly(vinyl acetate) (PVAC), poly(ethylene-co-vinyl acetate) (EVA), or poly(di-lactide-co-glycolide) (PLGA).

49. The method of claim 44 or 45, wherein the outer polymer skin is non-bioerodible.

50. The method of claim 44 or 45, wherein the polymer skin is non-bioerodible and is selected from polyurethane, polysilicone, poly(ethylene-co-vinyl acetate), polyvinyl alcohol, and derivatives and copolymers thereof.

51. The method of claim 45, wherein the polymer matrix is non-bioerodible.

52. The method of claim 51, wherein the non-bioerodible polymer matrix is selected from polyurethane, polysilicone, poly(ethylene-co-vinyl acetate), polyvinyl alcohol, and derivatives and copolymers thereof.

53. The method of claim 45, wherein the polymer matrix is bioerodible.

54. The method of claim 52, wherein the bioerodible polymer matrix is selected from polyanhydride, polylactic acid, polyglycolic acid, polyorthoester, polyalkylcyanoacrylate, and derivatives and copolymers thereof.

55. The method of claim 44 or 45, wherein the outer polymer skin is bioerodible.

56. The method of claim 44 or 45, wherein the outer polymer skin is bioerodible and is selected from polyanhydride, polylactic acid, polyglycolic acid, polyorthoester, polyalkylcyanoacrylate, and derivatives and copolymers thereof.

57. The method of claim 44 or 45, wherein sustained release of the at least one agent occurs for a period of at least 24 hours and, over that period of release, the concentration of the at least one agent in fluid outside the polymer skin is less than 10% of the concentration of the at least one agent in the polymer skin.

58. The method of claim 44 or 45, wherein sustained release of the at least one agent occurs for a period of at least 24 hours and, over that period of release, the concentration of the at least one agent in fluid outside the polymer skin is less than 5% of the concentration of the at least one agent in the polymer skin.

59. The method of claim 44 or 45, wherein sustained release of the at least one agent occurs for a period of at least 24 hours and, over that period of release, the concentration of the at least one agent in fluid outside the polymer skin is less than 1% of the concentration of the at least one agent in the polymer skin.

60. The method of claim 44 or 45, wherein sustained release of the at least one agent occurs for a period of at least 24 hours and, over that period of release, the concentration of the at least one agent in fluid outside the polymer skin is less than 0.1% of the concentration of the at least one agent in the polymer skin.

61. The method of claim 44 or 45, wherein the at least one agent and the outer polymer skin are co-extruded.

62. The method of claim 45, wherein the polymer matrix comprises a base having a pKa higher than the pKa of the at least one agent, or an acidic moiety having a pKa lower than the pKa of the at least one agent.

63. The method of claim 44 or 45, wherein the outer polymer skin comprises a base having a pKa higher than the pKa of the at least one agent, or an acidic moiety having a pKa lower than the pKa of the at least one agent.

64. The device of claim 44 or 45, wherein the at least one agent in its salt form is selected from timolol maleate, betaxolol hydrochloride, metformin hydrochloride, vancomycin hydrochloride, captopril, erythromycin lactobionate, ranitidine hydrochloride, sertraline hydrochloride, tramadol, ticlopidine hydrochloride, nicotine bitartrate, oxybutynin hydrochloride, diltiazem, propanolol, amoxicillin, cefuroxine axetil, cefaclor, clindamycin, azithromycin, ceftazidine, dorzolamide hydrochloride, acetazolamide, brinzolamide, methazolamide, and dichlorphenamide.

65. A method of treating glaucoma comprising:
providing a therapeutically effective amount of a free base of a highly water soluble salt of at least one agent that is effective in the treatment of glaucoma,
forming a sustained-release formulation having an inner core comprising the free base, wherein the inner core is covered at least in part by an outer polymer skin;
providing the sustained release formulation in a pharmaceutically acceptable carrier; and
administering the sustained release formulation to a patient having glaucoma.

66. The method of claim 63, wherein the inner core further comprises the free base dispersed in a polymer matrix.

67. The method of claims 62 or 63, wherein the agent is timolol maleate or betaxolol hydrochloride.
